(19) United States
(12) Patent Application Publication
(10) Pub. No.: US 2026/0174723 A1
(43) Pub. Date: Jun. 25, 2026

(54) INJECTABLE PACLITAXEL FORMULATION AND PREPARATION METHOD THEREOF

(71) Applicant: Riconpharma LLC, Denville, NJ (US)

(72) Inventors: Preetham Geejula, Hyderabad (IN); SVB Janardhan Garikipati, Hyderabad (IN); Vijaya Kumar Thota, Hyderabad (IN); Praveen Reddy Billa, Denville, NJ (US); Mukteeshwar Gande, Denville, NJ (US)

(73) Assignee: Riconpharma LLC, Denville, NJ (US)

(21) Appl. No.: 18/991,893

(22) Filed: Dec. 23, 2024

(51) Int. Cl. A61K 31/337; A61K 9/00; A61K 9/08; A61K 47/10; A61K 47/12; A61K 47/22

(52) U.S. Cl. CPC A61K 31/337; A61K 9/0019; A61K 9/08; A61K 47/10; A61K 47/12; A61K 47/22

(57) ABSTRACT

This invention relates to an injectable paclitaxel formulation designed to enhance solubility and stability. The formulation comprises paclitaxel at a concentration of 6 to 12 mg/mL, with Vitamin E Polyethylene Glycol Succinate (TPGS) serving as a surfactant and solubilizer in concentrations ranging from 300 mg/mL to 600 mg/mL. Polyethylene glycol 300 (PEG 300) acts as a stabilizer, and citric acid is included as a buffering agent, along with water for injection. The formulation is stable at room temperature for about 27 hours after dilution in infusion liquids such as 5% dextrose injection USP, 0.9% sodium chloride injection USP, 5% dextrose and 0.9% sodium chloride injection USP and 5% dextrose in Ringer's Injection solutions. The diluted formulation forms a nano micellar solution with micelles ranging from 10 to 100 nm in size, ensuring a stable and clear solution for intravenous administration of paclitaxel, thus improving safety and efficacy.

### FIELD OF THE INVENTION

This invention generally relates to pharmaceutical formulations and more specifically, a stable paclitaxel formulation that replaces Cremophor with Vitamin E Polyethylene Glycol Succinate (Tocopherol Polyethylene Glycol 1000 Succinate, abbreviated as “TPGS”) as a surfactant. The invention is designed to improve the solubility, stability, and safety profile of paclitaxel, particularly for intravenous administration.

### BACKGROUND OF THE INVENTION

Paclitaxel is one of the most commonly used anticancer drugs. The commercially available drug with paclitaxel is Taxol, which contains Cremophor EL (Polyoxyl 35 castor oil) as a surfactant to enable solubility in aqueous infusion liquid. Cremophor EL is proven to be associated with side effects, including hypersensitivity and neurotoxicity. Because of these reactions, paclitaxel is administered over 4 hours to minimize the hypersensitivity effects. The use of prophylactic steroids and histamine receptor antagonists as anti-allergic pre-medication may decrease the incidence and severity of acute hypersensitivity reactions. However, milder reactions have still been found to occur in 5%-30% of patients.

Abraxane is an albumin-bound paclitaxel nanosuspension product that is Cremophor free. Abraxane is available as a lyophilized powder which needs to be reconstituted to 5 mg/mL solution before dilution with infusion liquid. The main deficiency of the formulation is that they are complex formulations that are expensive and include a significant amount of albumin which can have the risk of microbial growth. Considering the formulation is available as the lyophilized powder, it includes an additional reconstitution step which adds complexity for administration and risk of contamination. Further, medication errors may arise due to the additional steps to prepare an infusion before administration. Although it reduces adverse reactions by eliminating Cremophor, it is a complex formulation and has the risk of microbial growth due to the presence of albumin.

The enhancement in solubility of paclitaxel by the use of hydroxyl propyl-β-CyD (HP-CyD) was demonstrated. It was postulated that due to the large taxane ring structure, the β and γ-CyD's with large cavities would be more suitable than the small α-CyD's for paclitaxel. The complexes formed with HP β-CyD were more stable than the complexes formed with HP γ-CyD or γ-CyD. However, it was observed that large amounts of CyD's were necessary to administer clinical doses of paclitaxel, which in turn causes significant renal toxicity and haemolysis (Journal of Pharmaceutical and Biomedical Analysis, vol. 13, pp. 533-541, 1995). Paclitaxel prodrugs were also evaluated as alternative options where the hydroxyl group at the C-2 position can be easily hydrolyzed by enzymatic or chemical means. Several paclitaxel C-2 esters, including succinate, glutarate, and sulfonic acid have improved water solubility and maintained in-vivo activity. However, they were shown to experience chemical instability in aqueous solution. The current RLD formulation for paclitaxel includes the paclitaxel dissolved in 49.7% ethanol and 52.7% polyoxyl 35 castor oil. Using polyoxl 35 castor oil is proven to be associated with a side effect, including hypersensitivity and neurotoxicity. Few trials were taken with surfactants, emulsifying agents, and cosolvents to improve the solubility and pharmacological properties of paclitaxel. However, the combination of these excipients can alter the absorption, distribution, metabolism, and excretion (ADME) of paclitaxel, leading to unpredictable pharmacokinetic profiles and making dosing more complex.

Accordingly, it is apparent that a need exists for a simpler, safer, and more stable formulation that maintains paclitaxel's efficacy while reducing the risk of adverse reactions.

### BRIEF SUMMARY OF THE INVENTION

The first aspect of the present invention provides an injectable paclitaxel formulation. The formulation includes paclitaxel at a concentration ranging from 6 to 12 mg/mL, Vitamin E Polyethylene Glycol Succinate (d-alpha-Tocopheryl Polyethylene Glycol 1000 Succinate or TPGS) as a surfactant and a solubilizer at a concentration ranging from 300 mg/mL to 600 mg/mL, polyethylene glycol 300 (PEG 300) as a stabilizer at a concentration ranging from 50 mg/mL to 500 mg/mL, citric acid as a buffering agent at a concentration ranging from 1 mg/mL to 5 mg/mL, and Water for Injection (WFI) at a range from 30 mg/mL to 100 mg/mL.

In an embodiment, the TPGS is present at a concentration preferably ranging from 500 mg/mL to 600 mg/mL.

In another embodiment, the PEG 300 is present at a concentration of 100 mg/mL.

In yet another embodiment, the citric acid is present at a concentration of 2 mg/mL.

In yet another embodiment, the formulation is chemically and physically stable at room temperature for about 27 hours after dilution with 5% dextrose Injection USP, 0.9% sodium chloride injection, USP, 5% dextrose and 0.9% sodium chloride injection, USP and 5% dextrose in Ringer's Injection solutions. The dilution concentrations are 0.3 mg/mL and 1.2 mg/mL.

In yet another embodiment, the formulation is diluted in infusion liquids to form a nano micellar solution with a micelle size ranging from 10 to 100 nm.

The second aspect of the present invention provides a method for preparing an injectable paclitaxel formulation. The method includes (i) dissolving Vitamin E Polyethylene Glycol Succinate (d-alpha-Tocopheryl Polyethylene Glycol 1000 Succinate or TPGS) at a concentration ranging from 300 mg/mL to 600 mg/mL in dehydrated alcohol to obtain a first solution, (ii) adding (a) polyethylene glycol 300 (PEG 300) at a concentration ranging from 50 mg/mL to 500 mg/mL, (b) Water for Injection (WFI) at a range from 30 mg/mL to 100 mg/mL, and (c) citric acid at a concentration ranging from 1 mg/mL to 5 mg/mL to the first solution to obtain a second solution; and (d) adding paclitaxel at a concentration ranging from 6 to 12 mg/mL to the second solution to obtain the injectable paclitaxel formulation.

In an embodiment, the TPGS is present at a concentration preferably ranging from 500 mg/mL to 600 mg/mL.

In another embodiment, the PEG 300 is present at a concentration of 100 mg/mL.

In yet another embodiment, the citric acid is present at a concentration of 2 mg/mL.

In yet another embodiment, the formulation is chemically and physically stable at room temperature for about 27 hours after dilution with 5% dextrose injection USP, 0.9% sodium chloride injection USP, 5% dextrose and 0.9% sodium chloride injection USP and 5% dextrose in Ringer's Injection solutions. The dilution concentrations are 0.3 mg/mL and 1.2 mg/mL.

In yet another embodiment, the formulation is diluted in infusion liquids to form a nano micellar solution with a micelle size ranging from 10 to 100 nm.

The formulation of the present invention eliminates Cremophor, reducing the risk of hypersensitivity reactions and neurotoxicity, which are common side effects of traditional paclitaxel formulations. The formulation includes TPGS, which has been approved by FDA as a safe adjuvant and is widely used in drug delivery systems replacing Cremophor as a surfactant, offering enhanced solubility and safety. The formulation allows for higher concentrations of paclitaxel, about 12 mg/mL, enabling smaller vial sizes and reducing storage requirements. The combination of PEG 300, water for injection, and citric acid enhances the chemical and physical stability of the formulation, ensuring its effectiveness over an extended period. The formulation of the present invention is also albumin-free which eliminates challenges with complex manufacturing and the presence of albumin. The formulation is a liquid product, eliminating the need for the additional reconstitution step required in the Abraxane product, thereby reducing the risk of medication errors.

### BRIEF DESCRIPTION OF THE DRAWINGS

A clear understanding of the key features of the invention summarized above may be had by reference to the appended drawings, which illustrate the method and system of the invention, although it will be understood that such drawings depict preferred embodiments of the invention and, therefore, are not to be considered as limiting its scope with regard to other embodiments which the invention is capable of contemplating. Accordingly:

FIG. 1 illustrates a method for preparing an injectable paclitaxel formulation according to various embodiments of the present invention.

Like reference numerals refer to like parts throughout the several views of the drawings.

### DETAILED DESCRIPTION OF THE INVENTION

The terminology used herein is for the purpose of describing particular embodiments only and is not intended to be limiting of the invention. As used herein, the term “and/or” includes any and all combinations of one or more of the associated listed items. As used herein, the singular forms “a,” “an,” and “the” are intended to include the plural forms as well as the singular forms, unless the context clearly indicates otherwise. It will be further understood that the terms “comprises” and/or “comprising,” when used in this specification, specify the presence of stated features, steps, operations, elements, and/or components, but do not preclude the presence or addition of one or more other features, steps, operations, elements, components, and/or groups thereof.

Unless otherwise defined, all terms (including technical and scientific terms) used herein have the same meaning as commonly understood by one having ordinary skill in the art to which this invention belongs. It will be further understood that terms, such as those defined in commonly used dictionaries, should be interpreted as having a meaning that is consistent with their meaning in the context of the relevant art and the present disclosure and will not be interpreted in an idealized or overly formal sense unless expressly so defined herein.

In describing the invention, it will be understood that a number of techniques and steps are disclosed. Each of these has individual benefit and each can also be used in conjunction with one or more, or in some cases all, of the other disclosed techniques. Accordingly, for the sake of clarity, this description will refrain from repeating every possible combination of the individual steps in an unnecessary fashion.

Nevertheless, the specification and claims should be read with the understanding that such combinations are entirely within the scope of the invention and the claims. An injectable paclitaxel formulation and preparation method thereof is discussed herein. In the following description, for purposes of explanation, numerous specific details are set forth in order to provide a thorough understanding of the present invention. It will be evident, however, to one skilled in the art that the present invention may be practiced without these specific details. The present disclosure is to be considered as an exemplification of the invention and is not intended to limit the invention to the specific embodiments illustrated by the figures or description below. The present invention will now be described by referencing the appended figures representing preferred embodiments.

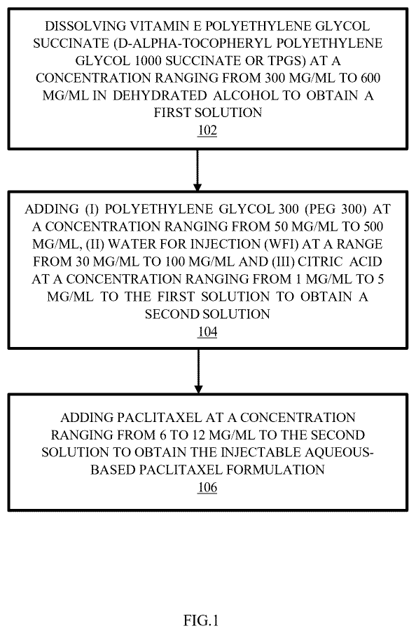

FIG. 1 illustrates a method for preparing an injectable paclitaxel formulation according to various embodiments of the present invention.

At step 102, the method includes dissolving Vitamin E Polyethylene Glycol Succinate (d-alpha-Tocopheryl Polyethylene Glycol 1000 Succinate or TPGS) at a concentration ranging from 300 mg/mL to 600 mg/mL in dehydrated alcohol to obtain a first solution.

At step 104, the method includes adding (a) polyethylene glycol 300 (PEG 300) at a concentration ranging from 50 mg/mL to 500 mg/mL, (b) Water for Injection (WFI) at a range from 30 mg/mL to 100 mg/mL, and (c) citric acid at a concentration ranging from 1 mg/mL to 5 mg/mL to the first solution to obtain a second solution.

At step 106, the method includes adding paclitaxel at a concentration ranging from 6 to 12 mg/mL to the second solution to obtain the injectable paclitaxel formulation.

### Example 1

Preparation of Paclitaxel Injection (12 mg/mL):

Approximately 66 g of the dehydrated alcohol was taken in a container, and 125 g of the Vitamin E TPGS was added and stirred to obtain a clear first solution. To the first solution, 25 g of the polyethylene glycol 300, 12.5 g of the water for injection and 0.5 g of weighed quantity of the citric acid anhydrous were added subsequently and stirred continuously to obtain a clear second solution. To the second solution, 3 g of the paclitaxel API was added under continuous stirring to form the injectable paclitaxel formulation. The volume was made up to 250 mL by using the dehydrated alcohol. The injectable paclitaxel formulation was passed through a 0.2 μm filter, and 8.35 mL of the filtered solution was dispensed into each vial, followed by stoppering with rubber stoppers and sealing with flip-off caps.

TABLE 1
Stability data for paclitaxel injection (12 mg/mL): Assay and impurity
profile under long-term and accelerated storage conditions.

| | | | Long Term Condition | | | Accelerated Condition | | |
|---|---|---|---|---|---|---|---|---|
| | | | (25° C./60% RH) | | | (40° C./75% RH) | | |
| Parameters | Limits | Initial | 1 Month | 3 Months | 6 Months | 1 Month | 3 Months | 6 Months |
| Assay | 90%-110% | 96.8 | 96.1 | 96.3 | 97.6 | 96.3 | 96.2 | 97.4 |
| Baccatin III | NMT 0.8% | ND | 0.01 | 0.03 | 0.02 | 0.01 | 0.02 | 0.05 |
| Ethyl ester | NMT 0.4% | ND | 0.01 | 0.02 | 0.01 | 0.01 | 0.02 | 0.01 |
| side chain | | | | | | | | |
| 10-Deacetyl | NMT 0.8% | 0.02 | 0.03 | 0.08 | 0.14 | 0.11 | 0.27 | 0.51 |
| paclitaxel | | | | | | | | |
| 10-Deacetyl-7- | NMT 0.5% | ND | ND | ND | 0.01 | ND | ND | 0.01 |
| epipaclitaxel | | | | | | | | |
| 7-Epipaclitaxel | NMT 0.6% | 0.06 | 0.06 | 0.06 | 0.06 | 0.07 | 0.06 | 0.07 |
| Highest | NMT 0.10% | 0.02 | 0.02 | 0.02 | 0.02 | 0.02 | 0.02 | 0.02 |
| Unknown | | | | | | | | |
| Total | NMT 2.0% | 0.10 | 0.26 | 0.27 | 0.42 | 0.30 | 0.56 | 0.79 |
| impurities | | | | | | | | |

The table 1 outlines the stability data of the paclitaxel formulation under long-term (25° C./60% RH) and accelerated (40° C./75% RH) storage conditions over six months. The assay results consistently fall within the acceptable range of 90%-110%, indicating stable potency across all conditions. Impurity levels show slight increases over time. For example, 10-Deacetyl paclitaxel rises from 0.02% initially to 0.51% at six months under accelerated conditions. Other impurities, such as Baccatin III and 7-Epipaclitaxel, also increase slightly but remain within their specified limits. Total impurities gradually rise from 0.10% initially to 0.79% after six months under accelerated conditions, staying well below the 2.0% threshold. Overall, the data indicates that the paclitaxel formulation maintains stability within acceptable impurity limits throughout the testing period.

### Example 2

Preparation of Paclitaxel Injection (6 mg/mL):

Approximately 142.4 g of the dehydrated alcohol was taken in a container, and 120 g of the Vitamin E TPGS was added and stirred to obtain a clear first solution. To the first solution, 40 g of the polyethylene glycol 300, 12 g of the water for injection and 0.8 g of weighed quantity of the citric acid anhydrous were added subsequently and stirred continuously to obtain a clear second solution. To the second solution, 2.4 g of the paclitaxel API was added under continuous stirring to form the injectable paclitaxel formulation. The volume was made up to 400 mL by using the dehydrated alcohol. The injectable paclitaxel formulation was filtered through a 0.2 μm filter, with 8.35 mL of the filtered solution filled into each vial, then stoppered with rubber stoppers and sealed with flip-off seals.

TABLE 2
Stability data for paclitaxel injection (6 mg/mL): Assay and impurity
profile under long-term and accelerated storage conditions.

| | | | Long Term Condition | | Accelerated Condition | |
|---|---|---|---|---|---|---|
| | | | (25° C./60% RH) | | (40° C./75% RH) | |
| Parameters | Limits | Initial | 1 Month | 3 Months | 1 Month | 3 Months |
| Assay | 90%-110% | 96.4 | 99.2 | 98.1 | 99.4 | 97.1 |
| Baccatin III | NMT 0.8% | ND | 0.01 | 0.01 | 0.01 | 0.03 |
| Ethyl ester side chain | NMT 0.4%. | ND | ND | ND | ND | ND |
| 10-Deacetyl | NMT 0.8% | 0.01 | 0.03 | 0.08 | 0.08 | 0.28 |
| paclitaxel | | | | | | |
| 10-Deacetyl-7- | NMT 0.5% | ND | ND | ND | ND | ND |
| epipaclitaxel | | | | | | |
| 7-Epipaclitaxel | NMT 0.6% | 0.05 | 0.06 | 0.06 | 0.06 | 0.08 |
| Highest Unknown | NMT 0.10% | 0.02 | 0.02 | 0.03 | 0.02 | 0.03 |
| Total impurities | NMT 2.0% | 0.12 | 0.23 | 0.25 | 0.25 | 0.52 |

The table 2 presents stability data of the paclitaxel formulation stored under long-term (25° C./60% RH) and accelerated (40° C./75% RH) conditions over three months. The assay results indicate that the active ingredient concentration remains within the acceptable range of 90%-110%, showing slight variations but staying consistent across both conditions. Impurity levels, such as Baccatin III and 10-Deacetyl paclitaxel, gradually increase over time, particularly under accelerated conditions. Despite these increases, all impurities, including 7-Epipaclitaxel and the highest unknown impurity, remain within their specified limits. The total impurities rise from an initial 0.12% to 0.52% under accelerated conditions after three months, still well below the 2.0% limit. Overall, the paclitaxel formulation maintains its stability within acceptable ranges across both storage conditions.

While the present invention has been described in terms of particular embodiments and applications, in both summarized and detailed forms, it is not intended that these descriptions in any way limit its scope to any such embodiments and applications. It will be understood that many substitutions, changes and variations in the described embodiments, applications and details of the method and system illustrated herein and of their operation can be made by those skilled in the art without departing from the spirit of this invention.

What is claimed is:

1. An injectable paclitaxel formulation, comprising:
paclitaxel at a concentration ranging from 6 to 12 mg/mL;
Vitamin E Polyethylene Glycol Succinate (d-alpha-Tocopheryl Polyethylene Glycol 1000 Succinate or TPGS) as a surfactant and a solubilizer at a concentration ranging from 300 mg/mL to 600 mg/mL;
polyethylene glycol 300 (PEG 300) as a stabilizer at a concentration ranging from 50 mg/mL to 500 mg/mL;
citric acid as a buffering agent at a concentration ranging from 1 mg/mL to 5 mg/mL; and
Water for Injection (WFI) at a range from 30 mg/mL to 100 mg/mL.

2. The formulation according to claim 1, wherein the TPGS is present at a concentration preferably ranging from 500 mg/mL to 600 mg/mL.

3. The formulation according to claim 1, wherein the PEG 300 is present at a concentration of 100 mg/mL.

4. The formulation according to claim 1, wherein the citric acid is present at a concentration of 2 mg/mL.

5. The formulation according to claim 1, wherein the formulation is chemically and physically stable at room temperature for about 27 hours after dilution with 5% dextrose injection USP, 0.9% sodium chloride injection USP, 5% dextrose and 0.9% sodium chloride injection USP and 5% dextrose in Ringer's Injection solutions, wherein the dilution concentrations are 0.3 mg/mL and 1.2 mg/mL.

6. The formulation according to claim 1, wherein the formulation is diluted in infusion liquids to form a nano micellar solution with a micelle size ranging from 10 to 100 nm.

7. A method for preparing an injectable paclitaxel formulation, wherein the method comprises:
dissolving Vitamin E Polyethylene Glycol Succinate (d-alpha-Tocopheryl Polyethylene Glycol 1000 Succinate or TPGS) at a concentration ranging from 300 mg/mL to 600 mg/mL in dehydrated alcohol to obtain a first solution;
adding (i) polyethylene glycol 300 (PEG 300) at a concentration ranging from 50 mg/mL to 500 mg/mL, (ii) Water for Injection (WFI) at a range from 30 mg/mL to 100 mg/mL, and (iii) citric acid at a concentration ranging from 1 mg/mL to 5 mg/mL to the first solution to obtain a second solution; and
adding paclitaxel at a concentration ranging from 6 to 12 mg/mL to the second solution to obtain the injectable paclitaxel formulation.

8. The method according to claim 7, wherein the TPGS is present at a concentration preferably ranging from 500 mg/mL to 600 mg/mL.

9. The method according to claim 7, wherein the PEG 300 is present at a concentration of 100 mg/mL.

10. The method according to claim 7, wherein the citric acid is present at a concentration of 2 mg/mL.

11. The method according to claim 7, wherein the formulation is chemically and physically stable at room temperature for about 27 hours after dilution with 5% dextrose injection USP, 0.9% sodium chloride injection USP, 5% dextrose and 0.9% sodium chloride injection USP and 5% dextrose in Ringer's Injection solutions, wherein the dilution concentrations are 0.3 mg/mL and 1.2 mg/mL.

12. The method according to claim 7, wherein the formulation is diluted in infusion liquids to form a nano micellar solution with a micelle size ranging from 10 to 100 nm.
